(19) United States
(12) Patent Application Publication
(10) Pub. No.: US 2026/0041851 A1
(43) Pub. Date: Feb. 12, 2026

(54) Dosage Verification System and Method for Safety Syringe

(71) Applicants: Thomas J. Shaw, Frisco, TX (US); Retractable Technologies, Inc., Little Elm, TX (US)

(72) Inventors: Thomas J. Shaw, Frisco, TX (US); Kathryn Duesman, Pilot Point, TX (US); Mark Small, Heavener, OK (US); Ni Zhu, McKinney, TX (US)

(21) Appl. No.: 19/359,907

(22) Filed: Oct. 16, 2025

Related U.S. Application Data
(62) Division of application No. 17/929,169, filed on Sep. 1, 2022, now Pat. No. 12,458,761.

(51) Int. Cl. A61M 5/315; A61M 5/31; G08B 21/18; G16H 20/17

(52) U.S. Cl. CPC A61M 5/31578; A61M 5/31535; A61M 5/31545; G08B 21/182; G16H 20/17; A61M 2005/3126; A61M 2205/18; A61M 2205/3569; A61M 2205/3592

(57) ABSTRACT

A dosage verification system for use prior to administering an injection of a medicinal fluid drawn into the fluid chamber of a medical syringe, the system comprising a medical syringe having a barrel with an outwardly facing, flat indicia display surface, a plunger having a plunger seal disposed inside and slidably engaging an inwardly facing wall of the barrel and a plunger handle projecting rearwardly from the barrel, and a hypodermic needle projecting forwardly from the barrel, all in combination with an imaging device configured to view and selectively capture, store or transmit a digital image of the plunger position relative to the barrel when a dose of the medicinal fluid is drawn into the fluid chamber of the barrel prior to an injection to verify and provide a record of the dosage drawn, and to alert a user prior to injection when an incorrect dosage is drawn into the fluid chamber of the syringe.

### CROSS-REFERENCE TO RELATED APPLICATIONS

This application is a divisional application of and claims priority to U.S. patent application Ser. No. 17/929,169 filed on Sep. 1, 2022 by Thomas J. Shaw et al., and entitled “Dosage Verification system and Method for Safety Syringe,” the disclosure of which is hereby incorporated by reference in its entirety.

### TECHNICAL FIELD

This invention relates to a system useful for quickly and reliably providing visual verification of medicinal doses drawn into a medical syringe prior to injecting a medication or vaccine into a patient or into another liquid infusion device. The subject system can be used to verify a dosage prior to an injection and to create a reliable visual record of the dosage administered if a question arises after an injection as to whether the dosage administered was correctly drawn or possibly constituted a medical error. One aspect of the subject system relates to a portable imaging device used in combination with a specially configured medical syringe to capture and record a digital image showing the volumetric scale, and preferably the whole volumetric scale, of a medical syringe and the position of the plunger and plunger seal inside the barrel of the syringe after drawing a dose of medicinal fluid. Another aspect of the invention relates to a medical syringe comprising a transparent or translucent barrel having at least one flat side forming an indicia display surface and a volumetric scale with numeric indicia disposed on the indicia display surface and overlying a fluid chamber extending longitudinally through the barrel to facilitate capturing and recording a digital image of the volume of a medicinal fluid drawn into the fluid chamber by a plunger slidably engaging an inwardly facing wall defining the circumferentially extending sidewall of the fluid chamber. Another aspect of the invention relates to a medical syringe comprising a barrel with a fluid chamber of known maximum useful volume in combination with a portable imaging system configured to capture a digital image of the syringe and determine the drawn dosage of a medicinal fluid from the position of the plunger handle relative to the barrel using ratio and proportion.

Still another aspect of the invention relates to a system comprising a medical safety syringe with a flat indicia display surface and a volumetric scale overlying a fluid chamber in combination with a digital imaging device configured to capture, record and optionally transmit a digital image of a medicinal fluid drawn into the fluid chamber to verify the dosage before administering the medicinal fluid to a patient. Still another aspect of the subject invention relates to the combination of a mobile digital imaging device such as a cellular telephone or digital tablet that is configured by application software resident in the device to enable a user of the device to capture, index and store digital images of a medical syringe containing a drawn dose of a fluid medication, generate a digital or documentary report, and trigger a visual, audible or tactile alert and/or alarm if the drawn dosage does not conform to a predetermined acceptable standard.

Still another aspect of the invention relates to a method for verifying the dose of a medicinal fluid drawn into a medical syringe prior to injecting the medicinal fluid into a patient or liquid infusion apparatus. As used in this disclosure, “medical syringe” refers to a medical device having an elongate barrel, generally made of a medical grade injection molded plastic, such as polypropylene, having a plunger with an elastomeric plunger seal slidably engaging an inwardly facing, cylindrical wall of the barrel that cooperate to define a fluid chamber disposed in fluid communication with a forwardly projecting hypodermic needle that is typically covered by a protective needle cap prior to use. As used in this disclosure, the term “safety syringe” is intended to refer to a medical syringe having the capability of causing the pointed tip of a hypodermic needle to be biased or retracted into a needle retraction cavity or to be covered with a protective guard or safety shield following use for the avoidance of accidental needle stick injuries.

### BACKGROUND OF THE INVENTION

Ways to identify and reduce the cause and occurrence of medical errors are a primary concern of the healthcare and insurance industries. A medical error is a preventable adverse effect of medical care, whether or not it is evident or harmful to the patient. Two commonly occurring sources of medical errors are the administration of the wrong drug or the wrong dosage of the right drug to a patient. Various protocols have been instituted in an effort to reduce such medical errors but they remain a principal concern. In many healthcare facilities, a medical caregiver must have another person verify the medicine and the drawn dosage prior to administering an injection to a patient. In situations where an adverse outcome arises during or following patient treatment, a verifiable record is needed to assist investigators in determining whether one causative factor was the dosage administered from a syringe by a healthcare worker. Similarly, in instances where injections are self-administered, such as with insulin injections, a verifiable record can assist in determining whether or not an incorrect dosage may have been a producing cause of an adverse outcome.

One difficulty that has arisen with the use of conventional medical syringes, and particularly with low-dose syringes such as 1 mL or 0.5 mL, is that the radius of curvature of the cylindrical barrels is so small that the volumetric indicia and associated numerals are difficult or impossible to print onto the barrel during manufacture and are also difficult to read when positioned so that they curve or wrap around the barrel. In many cases, and particularly where the medicinal fluid being injected is clear and colorless, a user may not be able to see and accurately determine the fluid dosage drawn into a syringe prior to an injection.

Another principal safety concern of healthcare institutions and medical caregivers are accidental needlestick injuries that can occur prior, during or subsequent to administering an injection. Such injuries are often preventable or controllable by appropriate product design and by strict adherence to safety protocols relating to the storage, use and post-use handling and disposal of needle products contaminated with blood-borne pathogens. A system is needed that can provide an accurate historical record of dosages drawn and administered with medical syringes while also effectively reducing the incidence and likelihood of accidental needle stick injuries.

### SUMMARY OF THE INVENTION

One embodiment of the invention disclosed here is a system comprising the combination of a medical syringe having a barrel with a flat, outwardly facing image display surface, a volumetric scale disposed on the flat image display surface overlying an elongate cylindrical fluid chamber inside the barrel, and a portable imaging device desirably configured to capture and store a digital image of at least a portion of the volumetric scale in relation to the dosage of a medicinal fluid drawn into the fluid chamber prior as viewed through a single wall of the barrel prior to administering an injection. In a preferred embodiment of the invention, the portable imaging device is a mobile device such as a cellular telephone or digital tablet equipped with a camera system that can focus on, capture and store a digital image of the entire volumetric scale of the medical syringe in a single frame in which the position of the plunger seal inside the barrel and the dosage level of a medicinal fluid present in the fluid chamber of the syringe barrel is also recorded. In another preferred embodiment of the invention, the digital imaging device is desirably configured and calibrated to record a digital image showing the position of a plunger handle relative to a syringe barrel comprising a fluid chamber of known volume from which the dosage level of a medicinal fluid drawn into the syringe can be calculated or otherwise determined.

In another preferred embodiment of the invention, the medical syringe is used in combination with a portable imaging device and also comprises a needle safety feature to reduce the likelihood of accidental needle sticks to patients, healthcare workers and subsequent handlers following use and during disposal of such syringes. One such needle safety feature comprises, for example, a needle retraction mechanism that biases and repositions the needle tip into a protected position relative to a needle retraction cavity following an injection. Another such needle safety feature comprises, for example, a needle safety shield that is forwardly slidable to cover the forwardly projecting tip of a hypodermic needle following an injection.

In one embodiment of the system of the invention, the imaging device is a cellular phone or digital tablet comprising application software downloadable from a proprietary service provider that is specially configured to scan, capture, record, display, store and index digital images of medical syringes for the purpose of determining and recording the dosage of medicinal fluid drawn into a syringe prior to administering an injection or infusion. Other ancillary components, functions and capabilities of such a system can be resident in the imaging device or provided as auxiliary components of the subject system. Such ancillary or auxiliary components an include a transmitter for forwarding digital images via wired or wireless connections; an optional comparator useful for determining whether doses of medicinal fluids drawn into a syringe are within permissible ranges or deviations from predetermined or preset values; an audio, visual or tactile alert generator signifying incorrect dosages; a digital display; a digital storage device or media; and a report generator useful for indexing and reporting digital data to be viewed, printed, or integrated into other electronic medical records. The dosage verification system can optionally be linked to a database of acceptable dosages for particular medicaments and patient profiles, and the dosage verification scans can, for example, be uploaded into digitized patient records accessible to various classes of identified user groups. Such user groups can include, for example, hospital risk managers, pharmacy managers, insurance managers, and the like.

According to one embodiment of the method of the invention, a dose of a medicinal fluid is drawn into the fluid chamber of a syringe, preferably a safety syringe, that is provided for use in combination with a portable digital imaging device to view through a single wall, capture, store and otherwise process a digital image of the dose drawn into the syringe. The digital image is useful for checking to see that the correct dosage of medicinal fluid has been drawn prior to injection and is also useful for providing an electronic or documentary record that can be used later to verify the dosage previously administered. A preferred syringe for use in practicing the subject method is a safety syringe comprising a barrel with an outwardly facing, flat, indicia display surface forming the outside portion of at least one side of an integrally molded, transparent polymeric wall that is desirably integrally molded together with the inwardly facing wall of an elongate cylindrical fluid chamber extending longitudinally through the barrel. In this embodiment, the method of the invention is practiced by drawing an intended dosage of a fluid medication (or vaccine) into a medical syringe; positioning the syringe so that the individual indicia and associated Arabic numerals of a volumetric scale disposed on a flat, outwardly facing indicia display surface of the syringe barrel are clearly visible in juxtaposition to the medicinal fluid level inside the syringe; positioning a portable imaging device so that a digital camera or scanner has a clear and unobstructed view of the medicinal fluid level inside the fluid chamber and of the volumetric indicia overlying the fluid chamber of the syringe; capturing a digital image of the medicinal fluid level inside the syringe; and saving the digital image in an archivable format that is recordable for use in verifying the dosage administered at a future time. In one variation of the subject embodiment of the method of the invention, the portable imaging device can be configured to compare the drawn dosage of the medicinal fluid to a predetermined value as soon as the image is captured and, if the drawn dosage is incorrect or outside an acceptable margin of error, trigger a visible, audible or tactile alert that will cause the user or medical caregiver to reassess the dosage being administered prior to giving the injection. In another variation of the subject embodiment, the image data can be merged into a template configured to generate a report suitable for inclusion in the appropriate medical records.

According to another embodiment of the method of the invention, a dose of a medicinal fluid is drawn into the fluid chamber of a medical syringe of known volume (e.g., 0.5 mL, 1 mL, 3 mL, etc.) that is provided for use in combination with a portable digital imaging device to view, capture, store and otherwise process a digital image of the plunger position relative to the barrel. In this embodiment of the invention, the portable imaging device is preferably configured by application software or other similarly effective means to determine the volume of the drawn dose of medicinal fluid from the position of the plunger handle relative to the barrel as compared to the plunger position when the plunger is positioned to utilize the full rated volume of the syringe. This method of determining the drawn dosage of a medicinal fluid by ratio and proportion can also be used with syringes not having a transparent barrel wall.

Other benefits and advantages of the subject system will likewise become more apparent to those of ordinary skill in the art upon reading this disclosure in relation to the accompanying drawings.

### BRIEF DESCRIPTION OF THE DRAWINGS

The system of the invention is further described and explained in relation to the following drawings wherein:

FIG. 1 is a top perspective view of an embodiment of the system showing a cellular telephone having a digital imaging capability disposed in a use position above a medical syringe containing a drawn dose of a medicinal fluid;

FIG. 2 is an enlarged top plan view of the cellular telephone of the system of FIG. 1 with a digital image showing a volumetric scale and numeric indicia disposed on an upwardly facing surface of syringe barrel overlying a fluid chamber containing a drawn dose of a medicinal fluid (shaded);

FIG. 3 is a top perspective view of another embodiment of the system showing a portably mounted digital imaging device having a digital imaging capability disposed in a use position above a medical syringe containing a drawn dose of a medicinal fluid;

FIG. 4 is an enlarged top plan view of the digital imaging device of FIG. 1 with a digital image showing a volumetric scale and numeric indicia disposed on an upwardly facing surface of syringe barrel overlying a fluid chamber containing a drawn dose of a medicinal fluid (shaded);

FIG. 5 is an exploded front perspective view of a medical syringe substantially as shown in FIGS. 1 and 3 but rotated 180° around the longitudinal axis so that the downwardly facing flat surface 17 of barrel 12 in FIG. 1 is facing upwardly in FIG. 5;

FIG. 6 is a longitudinal cross-sectional view of a medical syringe substantially as shown in FIGS. 1 and 3 with the needle cap removed and the plunger handle fully inserted into the barrel;

FIG. 7 is a top plan view of another safety medical syringe suitable for use in the system of the invention, with the needle cover in place, the plunger fully inserted into the barrel, and the forwardly slidable needle safety shield disposed in its fully retracted position;

FIG. 8 is a longitudinal cross-sectional view of the medical syringe of FIG. 7 with the needle cover removed to expose the forwardly extending needle tip, the plunger handle partially withdrawn to form a fluid chamber forwardly of the plunger seal, and the forwardly slidable needle safety shield still disposed in its fully retracted position as it would be during an injection;

FIG. 9 is another longitudinal cross-sectional view of the medical syringe of FIG. 8 with the plunger fully inserted into the barrel and the forwardly slidable needle safety shield moved to its forwardly extending position with the needle protection guard disposed around the forwardly extending tip of the needle as it would be following an injection; and

FIG. 10 is a diagrammatic representation of one embodiment of the system of the invention with an imaging device disposed in a use position above a medical syringe and also showing ancillary elements of the system to illustrate its desired functionality.

### DESCRIPTION OF PREFERRED EMBODIMENTS

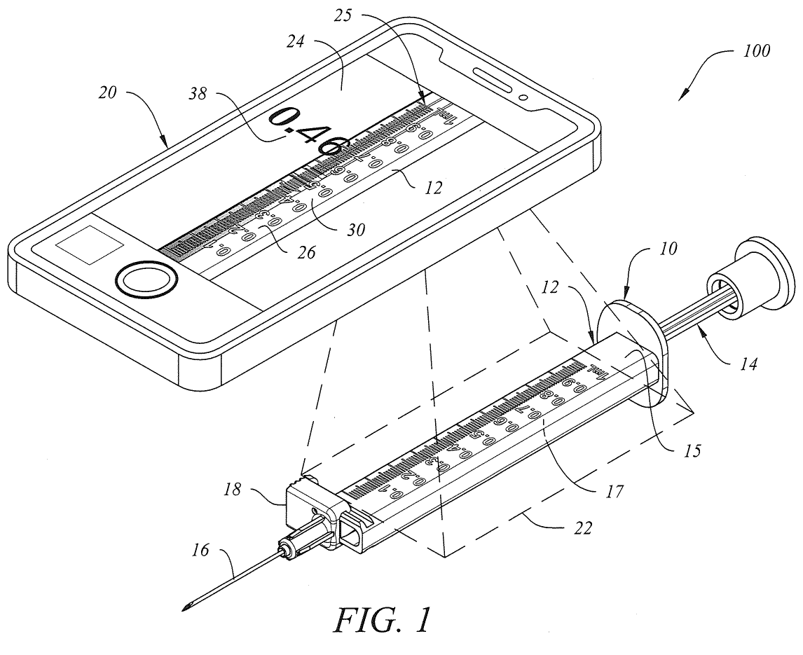

Referring to FIGS. 1, 2 and 5, one embodiment of dosage verification system 100 comprises a specially designed and configured safety medical syringe 10 in combination with a mobile imaging device 20 configured to capture a digital image display window 24 from a viewing field 22. It will be appreciated that the dimensions of viewing field 22 can vary in accordance with the spacing between safety medical syringe 10 and mobile imaging device 20 and with the lenses, focal lengths and circuitry of mobile imaging device 20. In one preferred embodiment as shown in FIG. 1, viewing field 22 desirably covers the full length of volumetric scale 25 disposed on flat, outwardly facing, indicia display surface 15 of barrel 12 of safety medical syringe 10.

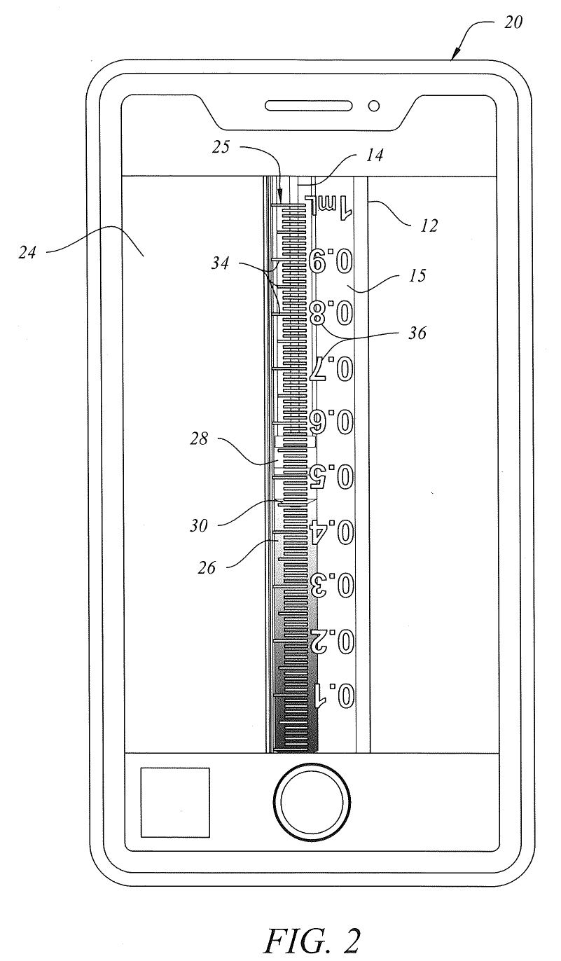

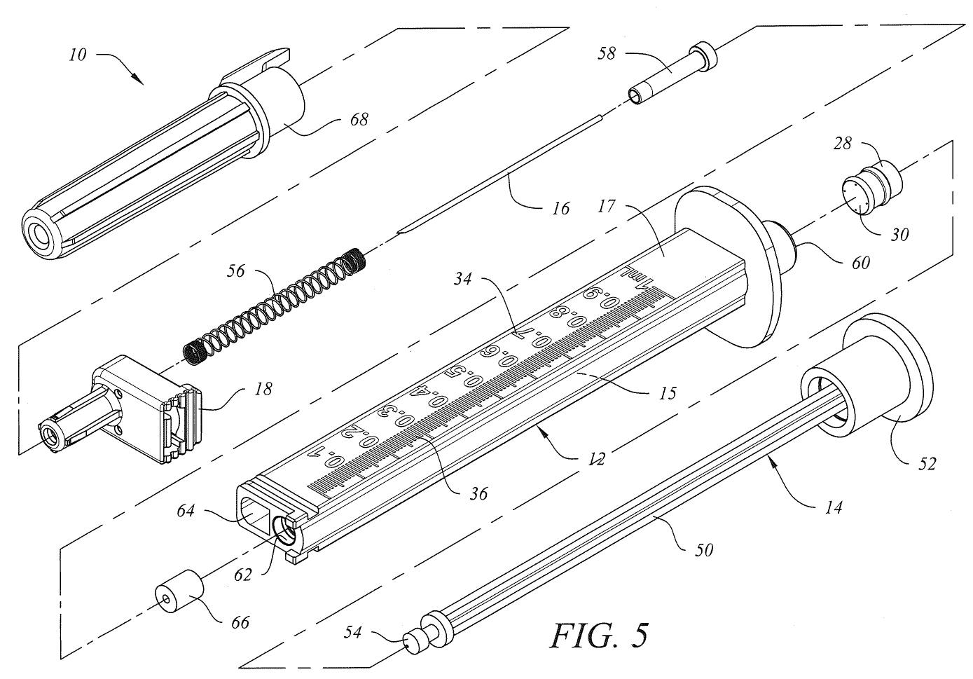

Barrel 12 is desirably unitarily molded from a transparent or translucent medical grade polymer and comprises a volumetric scale 25 having incremental markings 36 and accompanying numerical indicia 34 disposed on flat, outwardly facing indicia display surface 15 (oppositely facing indicia display surface 17 in FIG. 5) to overlie and facilitate simultaneous viewing of a fluid dosage 26 disposed inside longitudinally extending, cylindrical fluid chamber 62 (FIG. 5) that is slidably engaged and bounded rearwardly by the forwardly facing surface 30 of plunger seal 28 mounted on forwardly extending projection 54 of plunger handle 50 of plunger 14. Barrel 12 further comprises a forwardly projecting hypodermic needle 16 that is coaxially aligned with longitudinally extending, cylindrical fluid chamber 62. Hypodermic needle 16 is rearwardly biased inside transversely slidable frontal attachment 18 prior to an injection. As discussed in greater detail below, cylindrical fluid chamber 62 inside barrel 12 has an inwardly facing wall that is desirably unitarily molded with outwardly facing, flat indicia display surfaces 15, 17. Barrel 12 is desirably sufficiently transmissive to light that the forwardly extending end 30 of plunger seal 28 and medicinal fluid 26 drawn into fluid chamber 26 are clearly visible in digital image display window 24 (FIGS. 1 and 2) of mobile imaging device 20.

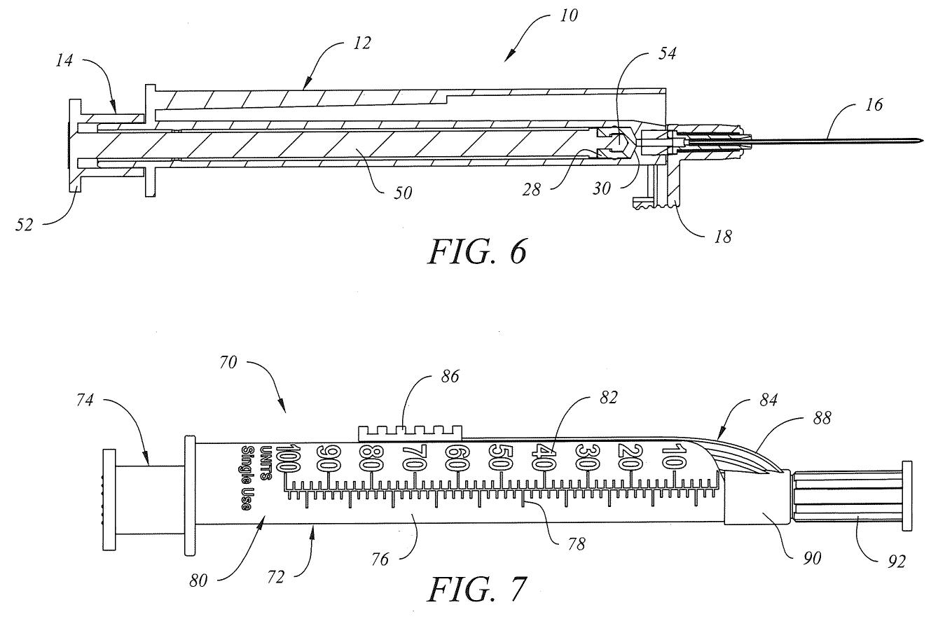

As depicted in FIGS. 1 and 2, mobile imaging device 20 is a cellular telephone with a camera system and imaging capabilities that are desirably configured by applications software to superimpose a digital readout of the fluid dosage drawn in medical syringe 10 as “0.46” mL. Grayscale shading in FIGS. 1 and 2 is intended to show for illustrative purposes the volume of medicinal fluid 26 drawn into the fluid chamber of medical syringe 10. In the enlargement depicted in FIG. 2, the drawn dosage of medicinal fluid 26 is more visible, with forwardly facing surface 30 of plunger seal 28 and a portion of handle 50 (FIGS. 5 and 6) of plunger 14 inside barrel 12 also being visible within the borders of digital image display window 24.

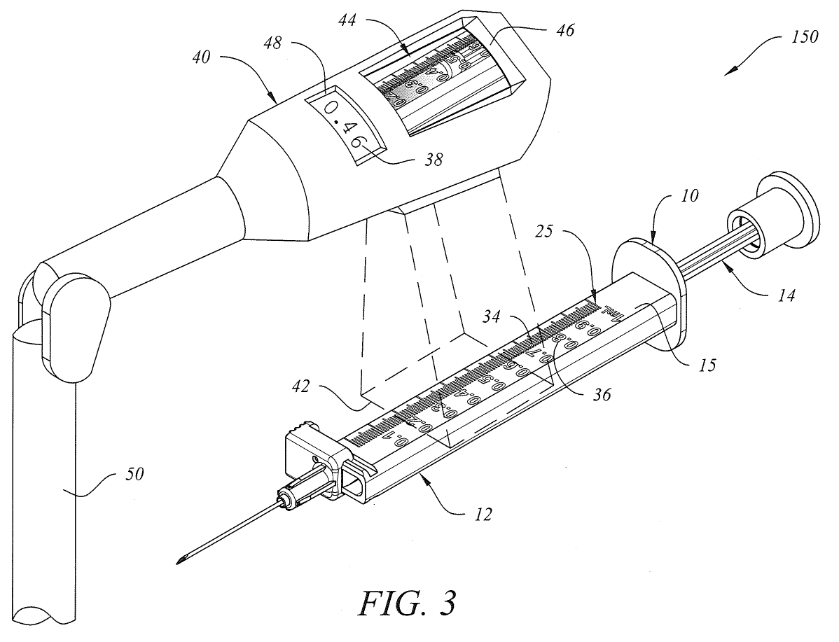

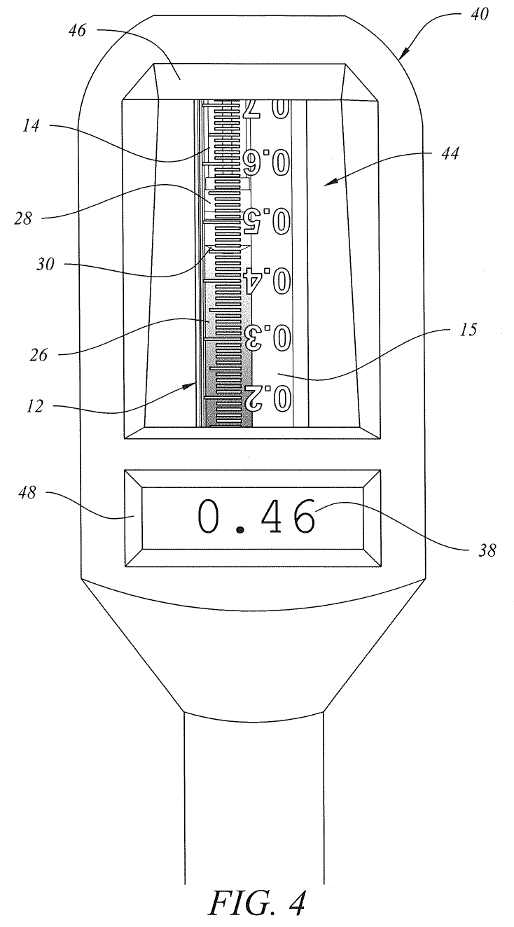

Referring to FIGS. 3 and 4, another embodiment of the invention identified as system 150 comprises a digital scanner 40 or other similarly effective, portable digital imaging device that can be mounted to a cart or bed frame in a hospital room or medical treatment area by apparatus such as hinged support rod 50 and adjustably positioned as needed to permit a user or medical caregiver to verify a dose of a medicinal fluid drawn into syringe 10. As previously shown and similarly described in relation to system 100, medical safety syringe 10 desirably comprises a barrel 12 further comprising an outwardly facing flat surface 15 having a volumetric scale 25 with individual indicia 34 and accompanying numerals 36 overlying the elongate fluid chamber inside barrel 12. As shown in FIGS. 3 and 4, digital imaging device 40 comprises a display screen 44 bounded by walls 46 that depicts the image framed within adjustable viewing field 42 focused upon upwardly facing, flat image display surface 15 of barrel 12 of medical syringe 10. Although not shown in FIG. 3, it should be understood that digital imaging device 40 is desirably operatively connected to a laptop or work station configured to capture, process, record, store and transmit digital images as needed to verify the dose of medicinal fluid drawn prior to an injection, trigger an audible, visual or tactile alert as needed if the drawn dosage is incorrect, and produce an archivable record of the dose administered in an injection for possible future use. Referring again to FIGS. 3 and 4, display 44 provides, in a single image, a clear and focused view of a dose of medicinal fluid 26 drawn into the fluid chamber of syringe barrel 12 forwardly of the front surface 30 of plunger seal 28 of plunger 14. Additionally, window 48 of digital imaging device 40 displays a digital readout of “0.46” (mL) as the volumetric dosage of medicinal fluid drawn into barrel 12 of medical syringe 10.

The structure and operation of safety medical syringe 10 is further described and explained in relation to FIGS. 5 and 6. Safety medical syringe 10 comprises unitarily molded barrel 12 having two longitudinally extending passageways including fluid chamber 62 defined by a cylindrical, inwardly facing wall and needle retraction chamber 64 and at least one outwardly facing, flat indicia display surface 17 having disposed on it a volumetric scale commensurate with the internal volume of fluid chamber 62. In the embodiment of FIG. 5, for example, there are two oppositely facing, flat indicia display surfaces 15, 17 the volumetric scale extends from 0.01 to 1.0 mL and comprises both individual markings 36 corresponding to 0.01 mL each and accompanying Arabic reference numerals 34 ranging from 0.1 to 1 mL. Plunger 14 comprising plunger handle 50, distal plunger tip 54 and proximal end cap 52 and an elastomeric plunger seal 28 attachable to distal plunger tip 54 is insertable into rear opening 60 of fluid chamber 62. Annular elastomeric fluid seal 66 is desirably seated inside the front opening of fluid chamber 62 prior to assembling frontal attachment 18 into transverse sliding engagement with the front of syringe barrel 12. Also seated in the forwardly projecting nose of frontal attachment 18 is a needle retraction assembly comprising a forwardly projecting hypodermic needle 16 disposed in fixed relation to elongate needle holder 38 and a compressed retraction spring 56 (shown in its uncompressed state in FIG. 5) that biases needle holder 58 and hypodermic needle 16 rearwardly prior to and during use of medical syringe 10. Needle cap 68 is a protective cap removed by a user prior to drawing a dose into fluid chamber 62 of syringe barrel 12 and should not be used to recap needle 16 following use to avoid needle stick injury to the user.

Referring to FIGS. 5 and 6, medical syringe 10 is depicted in its fully assembled form with plunger handle 50 and plunger seal 28 fully inserted into barrel 12. Needle cap 68 (FIG. 5) is removed to place medical syringe 10 in condition for drawing a dose of a medicinal fluid from a vial (not shown) into the fluid chamber 62 (FIG. 5). Medicinal fluid can enter fluid chamber 62 in front of front surface 30 of plunger seal 28 as plunger cap 52 of plunger 14 is pulled rearwardly after hypodermic needle 16 has been inserted into a vial of medicinal fluid or vaccine. As shown in the embodiment of FIGS. 5 and 6, the safety feature of safety syringe 10 is activated by applying transverse digital pressure to a touchpad of frontal attachment 18, thereby moving hypodermic needle 16 laterally from being coaxially aligned with fluid chamber 62 of barrel 12 to being coaxially aligned with an opening into needle retraction cavity 64 (FIG. 5). This lateral repositioning allows spring 56 to expand, driving needle holder 58 rearwardly into needle retraction cavity and also propelling the front tip end of hypodermic needle 16 rearwardly to a position where the needle tip is no longer exposed and cannot cause an accidental needle stick.

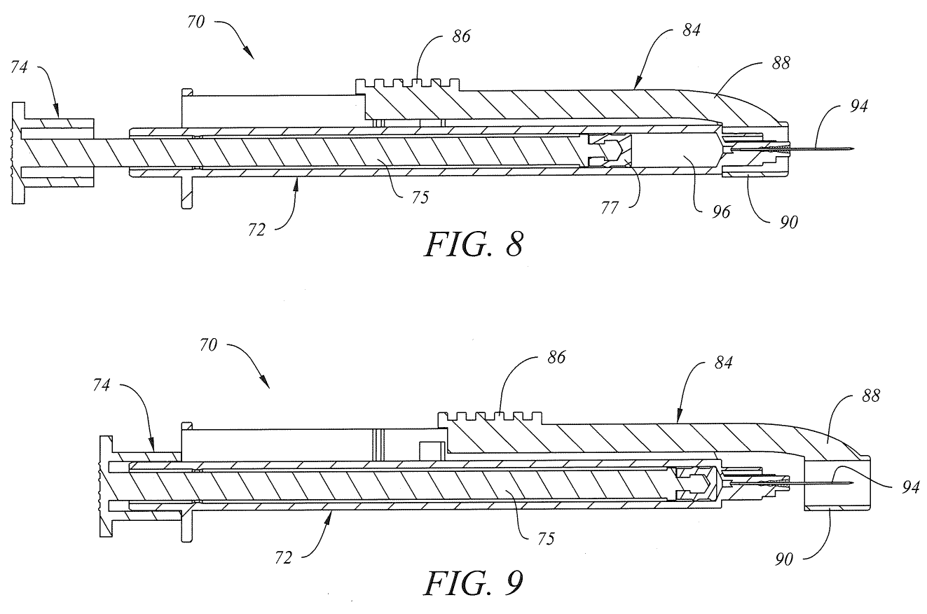

Another medical safety syringe 70 useful in practicing the system and method of the invention but having a different type of needle safety device is shown in FIGS. 7-9. FIG. 7 shows medical safety syringe 70 prior to use, with needle cap 92 in place. Barrel 72 further comprises an integrally molded, outwardly facing, flat indicia display surface 76 through which fluid chamber 96 (FIG. 8) is visible forwardly of plunger seal 77 when handle 75 of plunger 74 is partially withdrawn relative to barrel 72 when a dose of medicinal fluid is drawn into fluid chamber 96 of barrel 72 of syringe 70. Referring again to FIG. 7, a volumetric dosage scale 80 comprising incremental indicia 78 and accompanying numerical indicia 82 for a single use, 100-unit syringe is disposed on flat indicia display surface 76. Referring to FIGS. 7-8, a slidably engaged needle safety shield 84 having a touch surface 86, actuator handle 88 and circumferentially extending needle guard 90 are depicted in the fully retracted position that leaves the forwardly extending tip of hypodermic needle 94 uncovered and exposed when needle cap 92 is removed prior to use of syringe 70. Referring to FIG. 9, plunger handle 75 of plunger 74 is depicted in the fully inserted position relative to barrel 72 and needle safety shield 84 has been moved forwardly by applying forward pressure to touch surface 86 so that actuator handle 88 causes circumferentially extending needle guard 90 to encircle and protect the forwardly extending tip of hypodermic needle 94 for the avoidance of accidental needle stick injuries.

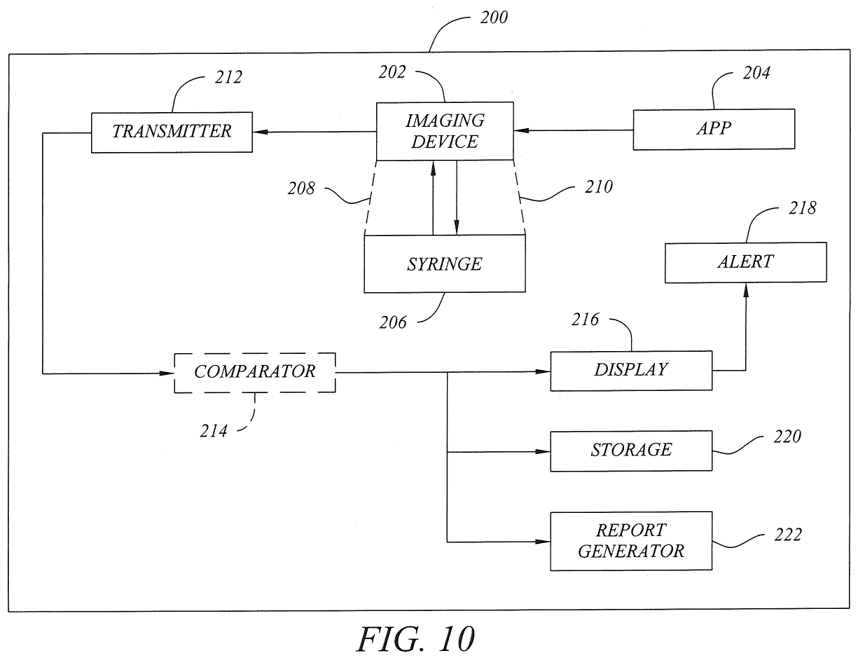

A simplified diagrammatic view illustrated in FIG. 10 is useful for further describing and practicing an embodiment of the invention identified as dosage verification system 200. Referring to FIG. 10, dosage verification system 200 includes a portable imaging device 202 positioned to view and capture a digital image of the upwardly facing surface of syringe 206 within a viewing field defined by boundaries 208, 210 that is sufficiently large to encompass the full range of a volumetric scale disposed on an upwardly facing, flat indicia display surface of syringe 206 as described previously. In one embodiment of the invention, portable imaging device 202 is configured by application software 204 to focus on and capture a clear image of the indicia display surface and volumetric scale (both individual indicia and accompanying numerals) of the syringe barrel in juxtaposition to the dose of a medicinal fluid drawn into the fluid chamber of the syringe barrel as previously described in relation to FIGS. 1-9 above. Application software 204 can be downloadable onto a mobile imaging device such as a cellular telephone having digital imaging capabilities or accessible by Wi-Fi, internet or other known technologies, and is desirably configured by use of a transmitter 212 or other conventional circuitry to control, process and communicate the digital images and digital image(s) viewed and captured by imaging device 202 to a display 216 and storage device or media 220 (that can be external or embodied in imaging device 202), or operatively linked to a report generator 222 or other archivable and indexed database. According to one embodiment of dosage verification system 200 of the invention, the digital images viewed and captured by imaging device 202 are optionally analyzed in relation to predetermined parameters by a comparator 214 or other similarly effective device or circuitry (with or without transmission to external devices) configured to generate a visual, audible or tactile alert 218 informing a user that the dose of medicinal fluid drawn into syringe 206 is more or less than the prescribed or intended dosage.

Other alterations and modifications of the invention will likewise become apparent to those of ordinary skill in the art upon reading this specification in view of the accompanying drawings, and it is intended that the scope of the invention disclosed herein be limited only by the broadest interpretation of the appended claims to which the inventors are legally entitled.

What is claimed is:

1. A method for administering an injection of a verified dosage of a medicinal fluid, comprising:
providing a syringe comprising a barrel having a fluid chamber and an outwardly facing flat indicia display surface, a plunger having a plunger seal slidably engaging the fluid chamber, and a forwardly projecting hypodermic needle;
providing a digital imaging device configured to selectively capture a digital image of the syringe;
drawing a dosage of the medicinal fluid into the fluid chamber;
positioning the digital imaging device relative to the syringe;
capturing the digital image of the syringe; and
processing the digital image to determine the dosage of the medicinal fluid drawn into the fluid chamber prior to administering the injection.

2. The method of claim 1, wherein the digital imaging device is a mobile imaging device.

3. The method of claim 2, wherein the digital imaging device is configured by downloading and installing application software on the digital imaging device.

4. The method of claim 3, wherein the application software is configured to capture the digital image of the syringe and process the digital image to determine whether the dosage of the medicinal fluid drawn into the fluid chamber is correct.

5. The method of claim 4, wherein the application software is configured to generate an alert to notify a user if a correct dosage or an incorrect dosage of the medicinal fluid has been drawn into the fluid chamber.

6. The method of claim 5, wherein the alert is at least one of an audio alert, a visual alert, and a tactile alert.

7. The method of claim 1, wherein the digital imaging device is configured to store the digital image of the syringe.

8. The method of claim 1, wherein the digital imaging device is configured to determine the dosage of the medicinal fluid in the fluid chamber by analyzing the position of the plunger with respect to the barrel.

9. The method of claim 8, wherein a volumetric scale is disposed on the outwardly facing flat indicia display surface.

10. The method of claim 9, wherein the digital imaging device is configured to determine the dosage of the medicinal fluid in the fluid chamber by analyzing a position of the plunger relative to the volumetric scale disposed on the outwardly facing flat indicia display surface.

11. The method of claim 1, wherein the digital imaging device is configured to determine the dosage of the medicinal fluid in the fluid chamber by analyzing the position of the plunger seal with respect to the barrel.

12. The method of claim 11, wherein a volumetric scale is disposed on the outwardly facing flat indicia display surface between a finger flange and a nose of the barrel.

13. The method of claim 12, wherein the digital imaging device is configured to determine the dosage of the medicinal fluid in the fluid chamber by analyzing a position of the plunger seal relative to the volumetric scale disposed on the outwardly facing flat indicia display surface of the barrel between the finger flange and the nose of the barrel.

14. The method of claim 1, wherein the medical syringe comprises at least one needle safety device configured to slide forwardly following an injection and surround a needle tip of the forwardly projecting needle to protect a user from an accidental needle stick.

15. A method for verifying a dosage of a medicinal fluid drawn into a fluid chamber of a barrel of a medical syringe prior to administering an injection with the medical syringe, comprising:
providing the medical syringe comprising the barrel having the fluid chamber and an outwardly facing flat indicia display surface at least partially overlying the fluid chamber of the barrel, a plunger having a plunger seal slidably engaging the fluid chamber, and a forwardly projecting hypodermic needle;
providing a digital imaging device configured to selectively capture a digital image of the syringe;
positioning the medical syringe within a field of view of the digital imaging device to provide a clear view of at least a portion of the outwardly facing flat indicia display surface and a full length of a volumetric scale disposed thereon and a position of the plunger with respect to the barrel;
capturing the digital image of the syringe of the position of the plunger relative to the barrel; and
processing the digital image to determine the dosage of the medicinal fluid drawn into the fluid chamber prior to administering the injection.

16. The method of claim 15, wherein the digital imaging device is configured to determine the dosage of the medicinal fluid in the fluid chamber by analyzing the position of the plunger with respect to the barrel, the full length of the volumetric scale disposed on the outwardly facing flat indicia display surface, or a combination thereof.

17. The method of claim 16, wherein the digital imaging device is configured to determine the dosage of the medicinal fluid in the fluid chamber by analyzing a position of the plunger relative to the volumetric scale disposed on the outwardly facing flat indicia display surface.

18. The method of claim 15, wherein the digital imaging device is configured to determine the dosage of the medicinal fluid in the fluid chamber by analyzing the position of the plunger seal with respect to the barrel the full length of the volumetric scale disposed on the outwardly facing flat indicia display surface between a finger flange and a nose of the barrel, or a combination thereof.

19. The method of claim 18, wherein the digital imaging device is configured to determine the dosage of the medicinal fluid in the fluid chamber by analyzing a position of the plunger seal relative to the volumetric scale disposed on the outwardly facing flat indicia display surface of the barrel between the finger flange and the nose of the barrel.

20. The method of claim 15, wherein the digital imaging device is configured by downloading and installing application software on the digital imaging device, wherein the application software is configured to capture the digital image of the syringe and process the digital image to determine whether the dosage of the medicinal fluid drawn into the fluid chamber is correct, and wherein the application software is configured to generate an alert to notify a user if a correct dosage or an incorrect dosage of the medicinal fluid has been drawn into the fluid chamber of the barrel.
